(19) United States
(12) Patent Application Publication
(10) Pub. No.: US 2018/0187233 A1
(43) Pub. Date: Jul. 5, 2018

(54) SOLARGAS SYSTEM OPERATED IN MULTIPLE MODES

(71) Applicant: Solaga UG, Berlin (DE)

(72) Inventors: David FEUSTEL, Berlin (DE); Benjamin HERZOG, Berlin (DE); Johann BAUERFEIND, Berlin (DE); Yannick BOLDT, Berlin (DE)

(73) Assignee: Solaga UG, Berlin (DE)

(21) Appl. No.: 15/738,552

(22) PCT Filed: Jun. 24, 2016
(86) PCT No.: PCT/EP2016/064650

§ 371 Date: Dec. 20, 2017

(30) Foreign Application Priority Data
Jun. 24, 2015 (EP) 15020102.8

(51) Int. Cl. C12P 39/00; C12P 7/42; C12P 5/02; C12M 1/107; C12M 1/00

(52) U.S. Cl. CPC C12P 39/00; C12P 7/42; C12P 5/023; C12M 21/04; C12M 29/22; C12M 21/02; C12M 29/04; C12M 29/20; C12M 23/22

(57) ABSTRACT

The invention relates to a method and a device for producing biogas in a combined fermenter, in the first section (2) of which organic material is produced by phototrophic microorganisms (1) using atmospheric carbon dioxide and oxygen, said organic material being used to produce biomethane by means of methanogens (5) in a second section (4). The fermenter is operated in multiple modes and using special gassing and degassing methods such that an optimal gas supply corresponding to the requirements of the respective microorganisms and preferably also the absorption or the use of atmospheric carbon dioxide is ensured.

The invention relates to a method for separating organic material from the aerobic section of a combined fermenter and converting it into biomethane in the anaerobic section, and a device for carrying out the method.

The production of biogas from plants is a promising technology for achieving the energy transition. Thanks to its convenient storability and high energy content, biogas is one of the most effective energy carriers. However, since 2012, the construction of new biogas systems has nearly stagnated in pioneering countries such as Germany due to legal and spatial limitations (Fachverband Biogas—prognosis from June 2014 according to which the number of installed systems will only rise by 61 to 8005 in 2015). In addition to a high demand in arable land steadily increasing the cost of food agriculture and the high effort involved in growing and harvesting the plants as well as purifying the biogases, the energy-intensive central supply of the energy carrier to the population is problematic.

Compared to terrestrial plants, phototrophic microorganisms (PTMs), including, but not limited to, cyanobacteria, seagrass and seaweed, exhibit higher growth rates and a higher productivity per unit area (Dismukes et al. 2008; FHI report of 2011). The cultivation of PTMs is not in any conflict with food production since PTM cultivation does not rely upon the use of arable land. Instead, PTMs are extremely undemanding and can be cultivated in places which are not fit for agricultural and industrial use, such as on impervious surfaces, in coastal regions, on unproductive land, e.g. in the desert or on salt-water springs, which do not have a corresponding water quality. In the broadest sense, PTMs only require carbon dioxide, the sun and trace elements to live and grow. The independence thus created not least facilitates a decentral supply of cost-efficient energy to the population.

With an estimated more than 45,000 species, PTMs represent a biotechnological and economic potential which is still far from exhausted. Since, comparable to plants, they use carbon dioxide for synthesizing organic material which is then released upon combustion, energetic recycling of carbon dioxide is possible without contributing to further global warming. Pure fermentation of PTMs into biogas is not very effective since the biogas produced is contaminated with ammonia and hydrogen sulfide and the purification of the biogas and the preconditioning of the PTMs consume a large amount of energy. In contrast, it was found that non-growing PTMs synthesizing organic and/or inorganic material comparable to microscopic factories are more suitable for the production of pure biogas. They do not require any fertilizer and the energy consumption is low after growth in the production phase. Biogas is not produced by the PTMs themselves. However, they can produce intermediates which are further metabolized into biogas by a partner organism, wherein both processes combined are more effective that the production of an energy carrier by PTMs alone.

In a method known from DE102007031688A1, algae produce hydrogen and oxygen from carbon dioxide and water which is converted into methane by methanogenic bacteria in a further step. CN103571876A improved upon this method by regulating the interfering oxygen release from the algae. Both methods have the disadvantage that they first produce starch in an energy-intensive manner, from which hydrogen and oxygen are obtained subsequently. Energy production by photovoltaics has proven to be more efficient than energy production by photosynthesis relating to glucose synthesis (Blankenship, R. E. et al. 2011). Indeed, with exposure to natural light, the efficiency is only 1 to 2% (Barber, J. 2009). This is further aggravated by the energy loss involved in the conversion into hydrogen, leading to an efficiency of 0.005% for the comparable cyanobacteria. An efficiency of approx. 1% could only be achieved through genetic engineering (Masukawa, H. et al. 2012).

DE102010040440A1 relates to a bioreactor in which photosynthetically active microorganisms are supplied with carbon dioxide and oxygen and excrete glycolate which is separated by a membrane and serves as a substrate for methanogenic microorganisms under anaerobic conditions, which convert it into carbon dioxide and methane. The method has the disadvantage that there is no membrane which is exclusively selective for glycolate and that a stripping chamber is not enough to sufficiently reduce the oxygen content. Furthermore, it is not clear how the atmospheric carbon dioxide is introduced into the system. As such, in order to use of a gas mixing device, separate gas storages with the respective pure gas are required, the filling of which at least affects the energy balance. Other unsolved problems are the supply of nutrient gas and/or oxygen to the microorganisms and the nutrient supply for two different media in continuous operation. Finally, it is not taken into consideration that non-adapted methanogenic bacteria are attacked by organic acids. The subject-matter of the inventive device according to DE102009008601A1 is a device having three compartments, in which a gas exchange occurs between the first and second compartments via a gas-permeable and liquid-impermeable membrane to treat a biological liquid, and a liquid exchange occurs between the first and third compartments through a membrane.

The invention is to solve the problem that high partial oxygen pressures are generated during the production of organic material in a subsection of a fermenter, while in another subsection, which is in communication with the first one, such high partial oxygen pressures are to be avoided. Another problem which is to be solved is that carbon from the air is to be integrated into energy carriers in the context of a fermentation process without high energy consumption.

According to the invention, the objective is achieved by the method described in claim 1, wherein the device described in claim 11 is suitable for carrying out the method.

The invention is based on the scientific basis described in the following.

PTMs produce and secrete organic material, i.e. carbon compounds such as organic carboxylic acids, under certain physiological conditions. For example, Synechocystis sp. PCC 6803 with inhibited glycogen synthesis produces α-ketoglutaric acid and pyruvic acid under nitrogen deprivation (Carrieri, D. et al. 2015).

At an increased oxygen/carbon dioxide ratio in comparison to air, PTMs produce organic material of particular biotechnological interest in the Calvin cycle by which carbon dioxide is fixed under normal conditions and used to synthesize glucose for energy supply. This phenomenon is based on the fact that ribulose-1,5-bisphosphate carboxylase/oxygenase (RuBisCO) also accepts oxygen as an alternative to carbon dioxide, wherein the KM value for oxygen of 350 μMol/l is greater than that for carbon dioxide of 9 μMol/l. A long time ago, the oxygen content in the earth's atmosphere was still so low that this process did not yet play any role. Upon reaction with oxygen, not only 3-phosphoglyceric acid (3-PGA) is formed but also 2-phosphoglycolic acid (2-PG) which can no longer be used in the Calvin cycle or otherwise in the organism and must therefore be recycled by other biochemical reactions or secreted. The oxygen pathway used for the regeneration of carbon dioxide for the Calvin cycle is considered to be one of the most wasteful processes on earth. However, this fact is utilizable for the natural biotechnology forming the basis of the method according to the invention.

The handling of 2-PG, the first stable intermediate downstream of the Calvin cycle, is different among organisms. 2-PG is converted into glycolic acid (hydroxyacetic acid) by dephosphorylation, partially secreted (FIG. 11) and partially metabolized into glyoxylic acid (FIG. 10). The latter is in turn also partially secreted or metabolized further, partially generating hydrogen peroxide, glycine, L-serine, α-ketoglutaric acid with nitrogen donors or glutamic acid (Bauwe, H. 2011). Unlike cyanobacteria, the metabolic pathways in eukaryotic PTMs occur in different compartments. For the most part, 3-PGA is regenerated from 2-PG and reintroduced into the Calvin cycle, wherein carbon dioxide is released or “exhaled”. To continue the production of glycolic acid, the ribulose-1,5-bisphosphate pool must be replenished which can only be done by intermediate absorption of carbon dioxide. To produce organic substances, it is also possible to use phototrophic microorganisms which excrete lipids or other long-chain hydrocarbons (lose cell wall material), such as of the genus Botryococcus. Upon anaerobic degradation, the use of lipids results in an improved methane content.

Biomethane-producing archaea, so-called methanogens, use organic material and synthesize biomethane from it. They are subdivided into acetate-splitting (acetotrophic) and hydrogen-oxidizing (hydrogenotrophic) methanogens. The acetotrophic methanogens cleave methyl groups from organic material and reduce them to biomethane. For this process, they use the methanophenazine coenzyme. They include Methanosarcina, among others. As early as in 1973, it was shown that anoxic microorganisms can grow with glycolic acid as the only source of carbon dioxide alone (Kurz, W. G. W. et al. 1973; Edenborn, H. M. et al. 1985). Friedrich M. et al. 1991 and Friedrich M. et al. 1996 discovered two acetotrophic strains growing on glycolic acid and producing methane in a mixed culture, wherein: 4 C2H4O3 (glycolic acid)->3 CH4+5 Co2+2 H2O. Egli, C. et al. 1989 showed that methanogens can live on monochloro- and dichloroacetate as the carbon and energy source.

In contrast, hydrogenotrophic methanogens, such as Methanococcus, Methanobacterium and Methanopyrus, form methane by reduction of carbon dioxide with hydrogen into methane and water or by conversion of formic acid. The do not have the methanophenazine enzyme. Most methanogens require an anoxic, pH-neutral or slightly alkaline medium containing at least 50% of water. Waterbody sediments, overly wet soils like moors and rice fields, manure, liquid manure and the intestine of ruminants represent excellent biotopes for methanogens. Inhibitors for methanogens are organic acids, disinfectants and oxygen.

The chemical equation for the formation of biomethane in the course of the overall process according to the invention is: CO2+2H2O->CH4+2O2.

To acclimatize microorganisms to special media components, nutrient sources and metabolites from other microorganisms, the microorganisms are gradually adapted to the components. This is done in a turbidostat which reduces the supply of medium with the component to be acclimatized to and increases the supply of a medium better suited for growth upon a decrease in growth or population density. This system does not only exploit the fact that, for evolutionary reasons, certain microorganisms have metabolic pathways which they do not need in their respective preferred habitats. Also, stable strains are generated by mutations in the context of long-term projects. This method allows to adapt methanogens to organic material, in particular organic acids, as the only carbon or energy source without genetic engineering.

The method of the invention is more efficient than the production of glucose-based biofuels for which the efficiency of glucose synthesis is only 1-2% (Linder, H. 1998). Approximately 36% of incident solar energy are stored in ATP due to the photosynthetic light reaction since four Einstein of red light with 172 kJ each correspond to 688 kJ and are sufficient for the transport of 2 moles of electrons or 218.9 Id, thus generating 1 mole of ATP with 30.6 kJ (together 249.5 kJ) which is required for carbon fixation in the Calvin cycle. Together with the consumption of NADPH, the efficiency of the Calvin cycle with synthesis and secretion is approx. 35% (Nabors, M. W. et al. 2007). The methane formation by archaea is very efficient with approx. 70% (Bernacchi, S. et al. 2014). As a result, the total efficiency of the formation of biomethane according to the invention is approx. 25% of the incident solar energy. Hence, the efficiency is higher than with photovoltaics which only achieves 16%.

The solution of the invention is described in the following.

In the method according to the invention, in the production mode, PTMs in a first section produce organic material, in particular glycolic acid, under oxic conditions from carbon dioxide and oxygen and secrete it into a medium. For this, they use sunlight and the trace elements present in the medium. Their growth is controlled. They do not require any fertilizer to grow or other components needed for growth in the medium. The organic material is secreted in a natural way. It is generated in the cells using oxygen instead of carbon dioxide (right side in FIG. 8).

During the exchange mode, after the production of the organic material, which advantageously requires a light irradiation of 100 to 200 μE/m²·s over more than one hour, the medium containing the organic material is fed into a second section in which the organic material is converted into biomethane by methanogens under anoxic conditions. During the transition to the second section, the medium is degassed to remove oxygen to prevent the oxygen, which is harmful to the methanogens, from reaching the second section. The methanogens are not compromised at a residual oxygen content of up to 4%. For example, based on glycolic acid, an average of 0.24 ml of biomethane is converted per mg of glycolic acid. A buffer optionally ensures that the required pH range is maintained. In an overall fermenter cycle, i.e. in a cycle concerning both sections of the combined fermenter, the medium is regassed with oxygen when flowing back to the first section to reestablish the toxic conditions for the production of the organic material in the first section. Preferably, the medium, in which the PTMs, here exemplified by cyanobacteria such as Gloeothece 6909, and the methanogens such as Clostridia such as Synthrophobotulus glycolicus or FlGlyM (Friedrich et al. 1996) as well as Methanococcus maripaludis are contained, has at least the following composition:

| Media component | Quantity in g/l |
|---|---|
| KH₂PO₄ | 0.12 |
| K₂HPO₄ | 0.12 |
| NaNO₃ | 0.15 |
| KCl | 0.42 |
| NH₄Cl | 0.25 |
| CaCl₂ × 2 H₂O | 0.12 |
| Resazurin | 0.75 · 10⁻³ |
| Na₂S × 9 H₂O | 0.40 |
| NaHCO₃ | 3.75 |
| L-Cysteine-HCl × H₂O | 0.40 |
| EDTA | 0.005 |
| NaCl | 4.50 |
| MgCl₂ × 6 H₂O | 1 |
| Trace element solution SL-10 (acc. to Wolfe) | 1 ml |
| Vitamin solution (acc. to DSMZ medium 141) | 10 ml |
| HEPES | 3 |

The medium is derived from the components recommended by the DSMZ, Leibniz Institut—Deutsche Sammlung von Mikroorganismen and Zellkulturen GmbH, and the Bold's Basal Medium suitable for cyanobacteria (Stein, J. 1973).

In a preferred embodiment of the method according to the invention, while contact to the second section is interrupted, the medium is degassed to remove oxygen and is gassed with air from the environment during return, the air is then used by the PTMs for carbon assimilation (regeneration mode), and the medium is then degassed to remove air and gassed with oxygen during return. After consumption of the organic material in the second section and the consumption of carbon compounds serving as the starting materials for the production of the organic material in the PTMs and present, in particular, in the reductive pentose phosphate pathway, advantageously after twice to four times the time of light irradiation in the production mode, the regeneration mode is to be initiated advantageously. Its purpose is to replenish the C3 pool in the Calvin cycle (Lorimer, G. H. et al. 1981) and the PTMs' own carbon supply (FIG. 8). In the production mode, in addition to 2-PG, 3-PGA is produced at a higher oxygen content (FIG. 9) which is reintroduced into the Calvin cycle. However, in net terms, carbon is lost from the Calvin cycle with each RuBisCO reaction. To compensate for this loss, it is necessary to intermittently reduce the oxygen content in favor of carbon dioxide. Atmospheric air is best suited for this purpose, the carbon dioxide in which, contributing to global warming, is to consumed. In the presence of this carbon dioxide, it is converted into 3-PGA. One part of it is used for regenerating the C3 pool and the other part is used for synthesizing endogenic carbon compounds such as glucose to supply the organism (FIG. 8). In the regeneration mode, the contact with the second section is interrupted. The PTMs are illuminated since the enzymes of the Calvin cycle are only active upon illumination.

During transition from the production mode having a high relative oxygen content in the medium to the regeneration mode with air dissolved in the medium, the medium is preferably discharged from the first section and degassed to remove oxygen. Then, the medium is preferably gassed with air and fed back to the first section, establishing a partial reactor cycle. Here, the exchange with the second section is preferably interrupted.

The transition from the regeneration mode to the production mode is preferably performed in the partial reactor cycle with interrupted contact to the second section by degassing the medium to remove air and gassing it with oxygen.

In a preferred embodiment of the method according to the invention, the ratio of carbon dioxide to oxygen in the medium located in the first section is low, preferably 1:800 to 1:3000, in the production mode. This corresponds to the range in which the RuBisCO accepts more oxygen and the precursors of organic material are produced by the PTMs without the oxygen content having a toxic effect. In contrast to this, the ratio in the regeneration mode corresponds to the air composition, 1:500. The reduction of the carbon dioxide/oxygen ratio is achieved by degassing to remove the air and gassing with oxygen upon transition to the production mode.

In a preferred embodiment of the method according to the invention, in the exchange mode, the medium is gassed with a substitute gas from a substitute gas storage, in particular with carbon dioxide or nitrogen, after the oxygen degassing during the transition from the first section to the second section. Thereby, the methanogens do not come into contact with oxygen, but with a gas which is not harmful to them. In particular, the carbon dioxide can be recovered from the biogas and first be fed into the substitute gas storage. Nitrogen is also suitable as a substitute gas due to its inert character and is also ubiquitous in air. During the return flow prior to oxygen gassing, the medium is degassed to remove the substitute gas. In its degassed condition, the medium is able and forced to absorb the oxygen. Thus, high partial oxygen pressures can again be achieved in the first section. The recovered gas is again stored in the substitute gas storage such that it is not lost and can be reused for gassing.

In a preferred embodiment of the method according to the invention, the carbon dioxide is separated from the biogas produced in the second section by a biogas filter and stored in the substitute gas storage for gassing with the substitute gas as described. Moreover, upon excess accumulation of carbon dioxide in the substitute gas storage, the carbon dioxide can be used in the regeneration mode instead of air. The separation of biogas into biomethane and carbon dioxide is preferably accomplished using a hollow fiber contactor. In this process, carbon dioxide is separated from the gaseous fiber through the pores of the hollow fibers which are smaller than 0.05 μm, while the more slowly passing biomethane is retained in the hollow fiber and transferred. It can then be stored in a biomethane storage and readily used for energy production. What is particular advantageous is that the concentrations in toxic nitrogen compounds, such as nitrogen oxides and ammonia, and sulfur compounds, such as hydrogen sulfide, are negligible, preferably less than 0.1%.

In a preferred embodiment of the method according to the invention, filters, in particular hollow fiber contactors, are used for the gassing and degassing, the pore size being less than 0.1 μm, preferably less than 0.04 μm. The hollow fiber contactors to be used preferably exhibit high effectiveness and a low energy demand for gassing and degassing liquids. During degassing, the medium is passed through the hydrophobic hollow fibers, a large number of which are present at the start and end of the contactor in a bundled form (FIG. 7). The large number of fibers creates a very large surface. Due to the low pore size and the hydrophobicity, only the gas molecules pass through the membrane which is enhanced by negative pressure. This process is reversed during gassing, i.e. positive pressure forces the gas molecules into the liquid without liquid molecules being able to reach the other side. Based on their different properties, different contactors are used for carbon dioxide and oxygen, having the following hollow fiber characteristics:

| Membrane contactor | Carbon dioxide | Oxygen |
|---|---|---|
| External diameter (in μm) | 300 | 300 |
| Internal diameter (in μm) | 200 | 220 |
| Bubble point (in psi) | 240 | 240 |
| Porosity (in %) | 25 | 40 |
| Pore size (in μm) | 0.03 | 0.04 |

The membranes are provided by 3M Deutschland GmbH, for example. The hollow fiber contactors to be used preferably for degassing and gassing have an inlet and an outlet for the liquid flow. These are connected to an inlet space and an outlet space, respectively. Therebetween, the hollow fibers are located which are secured on both sides by a partition. The partitions delimit the permeate space through which the hollow fibers are tensioned. A pump is connected to one opening of the permeate space. During degassing, this is a gas pump extracting the gas from the hollow fibers, thus, from their perspective, a vacuum pump. In FIG. 7, for gassing, it is a gas pump feeding the gas to the hollow fibers and creating positive pressure in the permeate space. In particular, the use of hollow fiber contactors has the advantage of accomplishing an optimum supply of gas to the microorganisms since the gas can be dissolved best in the medium with their help, achieving a better and more energy-conserving supply than with conventional methods such as bubbling and mixing. Specifically, the use of hollow fibers can reduce the oxygen content to less than 3%.

In a preferred embodiment of the method according to the invention, cyanobacteria, in particular Gloeothece 6909, Plectonema boryanum, Anabaena sp. and Nostoc sp., are present as the PTMs in the first section. Cyanobacteria have proven to be very well suited for the method according to the invention. In contrast to green algae, which do not absorb within the so-called green gap, they can absorb light of all wavelengths by means of phycobilins and convert it into chemical energy (MacColl, R. 1998). With the phycobilin phycoerythrin, the efficiency of light utilization is even higher than with chlorophyll. This enables cyanobacteria to even successfully colonize low-light regions, such as found on the bottom of river pebbles or in lower layers of lakes, for example (Sari, S. 2010). Therefore, they can produce organic material in the production mode even in the event of low light intensities. Furthermore, cyanobacteria are preferred because they are insensitive to light and temperature, i.e. can withstand higher irradiation and lower temperatures (Latifi, A. et al. 2009). They are among the oldest creatures and have colonized almost every habitat on earth. In contrast to eukaryotic PTMs, they do not have any comparable cellular compartments which makes them relatively easy to analyze and manipulate. In addition, they are advantageous for the method according to the invention due their high production rates of organic material. For example, the species and genera mentioned exhibit high production rates and secretion rates of glycolic acid (Renström, E. et al. 1989). More preferred are biofilm-forming cyanobacteria. The species and genera mentioned form said biofilm in the first section, preventing them from being transported into the second section. More preferred are cyanobacteria metabolizing the produced organic material minimally. One strategy of the cyanobacteria to handle produced organic material is the secretion into the surrounding environment instead of the energy-intensive transformation into intermediates utilizable for them. Preferred are those species which do not metabolize more than 10% of the organic material. Many green algae species, such as Chlamydomonas reinhardtii, produce glycolic acid in a significant amount. However, they metabolize a large part of it (Moroney, J. V. et al. 1986) which is why they are less suitable for the method according to the invention. As a result, with the method according to the invention, a non-genetically modified technology can be used which can also be employed at a consumer level.

In a preferred embodiment of the method according to the invention, the methanogens in the second are a mixture of acetotrophic and hydrogenotrophic archaea. The acetotrophic archaea, in particular Methanosarcina sp. or Synthrophobotulus sp., split the organic material from the first section into carbon dioxide and hydrogen. It is then used by hydrogenotrophic archaea, in particular Methanocella paludicola, Methanocella arvoryzae or Methanopyrus kandleri, for biomethane production. Mixed cultures obtained by selection from sediments of lakes and oceans, bovine rumen, intestines of termites and other animals, rice fields, marshes or biogas systems are also preferably used in the second section. During selection, organic material is gradually added to the archaea, preferable in the turbidostat described above, until carbon is exclusively supplied by such material. For this purpose, the inventors used fermentation sludge from a conventional biogas system. The operating temperature varies between 37° C. and 70° C., wherein the gas production speed correlates with the temperature. This allows to set the production rate. In addition, waste heat from the fermenters can be used for heating the respective other fermenters.

In a preferred embodiment of the method according to the invention, inhibitors of intracellular degradation of the organic material and of intracellular carbon dioxide storage are present in the first section. Inhibitors of intracellular degradation of glycolic acid include, for example, 3-decyl-2,5-dioxo-4-hydroxy-3-pyrroline (Stenberg, K. 1997), 4-carboxy-5-(1-pentyl)hexylsulfanyl-1,2,3-triazole (Stenberg, K. 1997), butyl 2-hydroxy-3-butynoates (Doravari, S. et al. 1980) and α-hydroxy-2-pyridinmethanesulfonic acid (Zelitch, I. 1966), which primarily inhibit glycolic acid oxidase. Inhibitors of carbon dioxide storage include, but are not limited to, acetazolamide (Moroney, J. V. et al. 2001) and glycolaldehyde (Miller, A. G. et al. 1989), which suppress the conversion of carbon dioxide into bicarbonate, a form in which carbon dioxide is stored. Preferably, the inhibitors are added to the medium in the first section to optimize the production of organic material.

In a preferred embodiment of the method according to the invention, the expression and/or the activity of glycolic acid dehydrogenase (EC 1.1.99.14) and/or glycolic acid oxidase (EC 1.1.3.15) are suppressed in the PTMs or corresponding PTMs are selected using activity measurements. For example, the suppression is achieved through shRNA (small hairpin RNA), an RNA molecule having a hairpin structure which is able to artificially silence genes by RNA interference (RNAi). Alternatively, siRNAs (small interfering RNA), short single- or double-stranded ribonucleic acid molecules with a length of 20 to 25 base pairs, can be used. They capture complementary single-stranded RNA molecules and thus inhibit the expression of the associated protein. Still preferred, carbon dioxide accumulation by carboxysomes and pyrenoids is inhibited. For this purpose, the expression of carbonic anhydrase (EC 4.2.1.1.) required for carbon dioxide accumulation in the carboxysomes is suppressed by shRNA, siRNA or other means such as gene knockouts. Preferably, ribulose-1,5-bisphosphate carboxylase/oxygenase (EC 4.1.1.39) and/or glycolic acid phosphate phosphatase (EC 3.1.3.18) are overexpressed. The overexpression can be achieved by introducing a plasmid with the respective gene into the organism or by incorporating, optionally multiple instances of, an overexpression cassette with the respective gene into the organism's genome, the gene being flanked by a strong promoter in either case. RuBisCO is the enzyme directing the carbon dioxide/oxygen assimilation within the Calvin cycle, while glycolic acid phosphate phosphatase precedes the production of the secreted glycolic acid (FIG. 10). Thus, the expression of both enzymes leads to an enhanced production of glycolic acid. More preferred, RuBisCO of type II is expressed in the PTMs. The expression can be accomplished by introducing a plasmid or cassette into the genome. RuBisCO of type II, which is of bacterial origin, has a higher turnover number than RuBisCO of type I so that its incorporation is beneficial in terms of higher productivity. Preferably, the excretion of organic material, especially of glycolic acid, through the cell membrane is enhanced. This is achieved through overexpression or enhanced activation of transport proteins in the cell membrane which are responsible for the transport of the relevant organic material. With the cyanobacteria to be used advantageously, targeting the transporters in compartmental membranes is not required as prokaryotes do not have compartments corresponding to the other PTMs, while this is necessary with eukaryotic PTMs.

The device according to the invention for producing biogas in a combined fermenter comprises a first section, which is partially transparent, with phototrophic microorganisms located in a medium. The transparency is ensured by using transparent materials such as glass, forms of acrylic glass (polymethylmethacrylates), polycarbonate, polyvinylchloride, polystyrene, polyphenylenether and polyethylene.

Preferably, the first section is a flat module comparable to a solar panel, since the depth of the first section within the axis along solar irradiation is limited by the maximum illumination depth. According to the invention, the PTMs are immobilized such that more than 90% of the incident solar irradiation are absorbed, wherein a depth of no more than 10 mm is not exceeded. The modules are suitable for convenient installation on open spaces or building tops and can be combined with each other and extended in any manner. At the end of the PTMs' lifespan, they can be exchanged and fed into conventional biogas systems as an additive to increase yield.

Moreover, the device comprises a second section, which is opaque, with methanogens located in the medium which is exchanged in exchange mode with the first section, the oxygen content being reduced. Another component is a connecting system between the first section and the second section, in which the exchange and gassing and degassing of the medium are performed using filters, wherein the filters are connected to gas feeds and gas discharges. Another component is a biogas discharge from the second section to discharge the biogas produced. Pumps and valves are used for distributing the liquid and gas streams corresponding to the embodiments illustrated by way of example with reference to the drawings. The further design of the device is determined by the advantageous methods described above. Preferably, both sections are connected to containers containing nutrients, such as trace elements, or used for discharging by-products. Sensors can be used for process control. The materials of both sections other than the transparent region in the first section are metals and plastics, for example, as required.

In a preferred embodiment of the device according to the invention, the connecting system comprises first and second connecting pipes to enable establishing a full reactor cycle in the exchange mode. Moreover, the connecting system preferably comprises a cross-connecting pipe, interconnecting the first connecting pipe and the second connecting pipe. This allows to establish a partial reactor cycle for the transitions between the regeneration and production modes. Still preferably, filters are provided as parts of the connecting pipes between the sections and the cross-connecting pipe. These filters are used for gassing and degassing in the exchange mode (first to fourth filters) and in the transitions from the exchange mode to the regeneration mode and from the regeneration mode to the production mode (first and second filters). Moreover, the device preferably comprises a second cross-connecting pipe connecting the first section and the cross-connecting pipe, and a fifth filter as a part of the second cross-connecting pipe having an air line with an air filter, preferably with a pore size of 0.2 μm or less, to sterilize the air drawn in. The fifth filter is used for gassing and degassing with air in the transitions from the exchange mode to the regeneration mode and from the regeneration mode to the production mode, wherein the first and second filters are responsible for their respective gassing and degassing functions with oxygen. Preferably, the fermenter has an oxygen discharge, an oxygen feed, a substitute gas discharge and a substitute gas feed, each connected to a gas pump and the respective filter, as well as a substitute gas storage or an oxygen storage, each connected to the substitute gas and oxygen lines, respectively. This way the gases can be stored intermediately after combined gassing and degassing operations. Still preferably, the biogas discharge passes from the second section to a biogas filter and then to a biomethane storage tank. Preferably, the biogas filter is connected to the substitute gas storage. This way, the carbon dioxide extracted by the biogas filter can be fed back to the substitute gas cycle. It is also contemplated to provide excess carbon dioxide in the regeneration mode. As a result, this constitutes a dual cycle. In the first section, a pH sensor or conductivity sensor is preferably used to assess how high the percentage of organic acids is in the medium and when the exchange mode must be initiated correspondingly. Alternatively, solar sensors can be employed to assess the activity of PTMs.

In a preferred embodiment of the device according to the invention, the first section is protected from excessive solar irradiation. The purpose of this is to protect the PTMs whose photo systems could be damaged by excessive solar irradiation and/or which are endangered by the release of harmful reactive oxygen species. Specifically, the transparent region of the first section may be gradually darkened. For example, electrochromic materials can be used in the transparent region of the first section.

In a preferred embodiment of the device according to the invention, the phototrophic microorganisms are preferably immobilized on cellulose or a mobile carrier material, in particular in gas-permeable gel capsules, and the methanogens are preferably immobilized on activated carbon and/or a mobile carrier material, in particular in gas-permeable gel capsules. The purpose of the immobilization is to retain the PTMs and methanogens in their respective sections. Only the medium is to be exchanged. Possible immobilization techniques include (covalent) binding to the surface, filling of pores, cross-linking, membrane separation and immobilization by inclusion. For example, the preferably biofilm-forming PTMs can be grown onto a carrier surface such as cellulose, alginate, chitosan or agar. Movable carriers are also contemplated, which are retained at the outlet of the section by a filter and thus contribute to immobilization in the relevant section. In addition, so-called immobilization tags are possible, i.e. proteins which are recombinantly expressed on the surface of microorganisms and provide for binding to a carrier material thereon. Advantageously, the methanogens may also be immobilized on activated carbon.

Advantageous effects of the invention compared to the prior art and also with respect to industrial application become apparent from the description of the method according to the invention and the device according to the invention.

The advantageous effects of the invention compared to the prior art include an optimum, especially energy-conserving transfer of the organic material from an aerobic section of the combined fermenter to an anaerobic section. This helps to achieve an improved aeration of the microorganisms without unnecessary gas consumption. In addition, in a preferred variant, there is the possibility to assimilate carbon dioxide (and nitrogen) from the air and thus initiate a climate- or CO2-neutral process leading to the release of carbon dioxide during biomethane combustion. As a result, the method according to the invention in the intended device is more effective, especially when compared to the production of biogas from plants, not only relating to the lesser use of arable land and the high costs involved in the production of biomass:

| | Biogas yield m³/t | Methane |
|---|---|---|
| | biomass (organ. mat.) | content in% |
| Corn | 198 | 54 |
| Grass | 158 | 52.9 |
| Euk. PTMs | 400 (0.24 ml/mg of glyc. · 5/3 · 10³) | 37.5 |

Conv. biogas numbers from Schwab, M. 2007

### DESCRIPTION OF THE DRAWINGS

FIG. 1 shows a two-dimensional view of a device according to the invention for carrying out the method, wherein dashed parts represent a level transition.

FIGS. 2-6 show views of the fermenter shown in FIG. 1 in multiple modes.

FIG. 7 shows the layout of a hollow fiber contactor preferably used as a filter.

FIG. 8 schematically shows the sequence of the Calvin cycle in phototrophic microorganisms under carbon-dioxide-rich conditions (left side) and under oxygen-rich conditions (right side).

FIG. 9 shows the reactions catalyzed by RuBisCO with the molecules involved indicated as structural formulas.

FIG. 10 shows the oxygenase reaction of RuBisCO and the further metabolization of the phosphoglycolic acid thus produced with the molecules involved indicated as structural formulas and the enzymes.

FIG. 11 shows a diagram on the production of glycolic acid in μmol per mg of chlorophyll a by Gloeothece 6909 at different oxygen/carbon dioxide ratios and different irradiances.

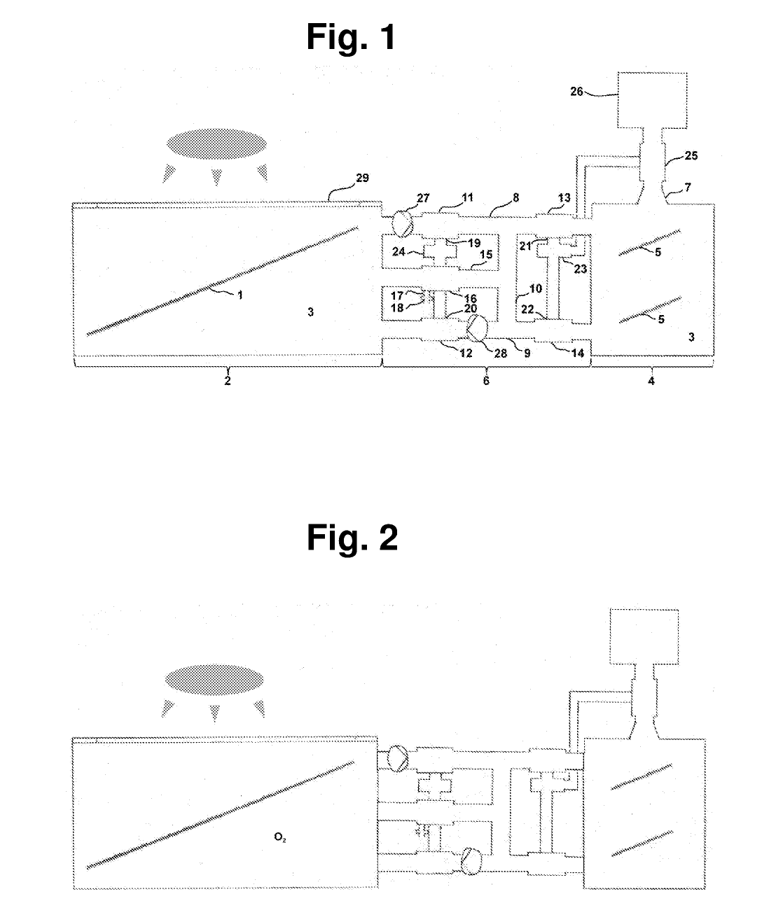

### DESCRIPTION OF AN EXEMPLARY EMBODIMENT

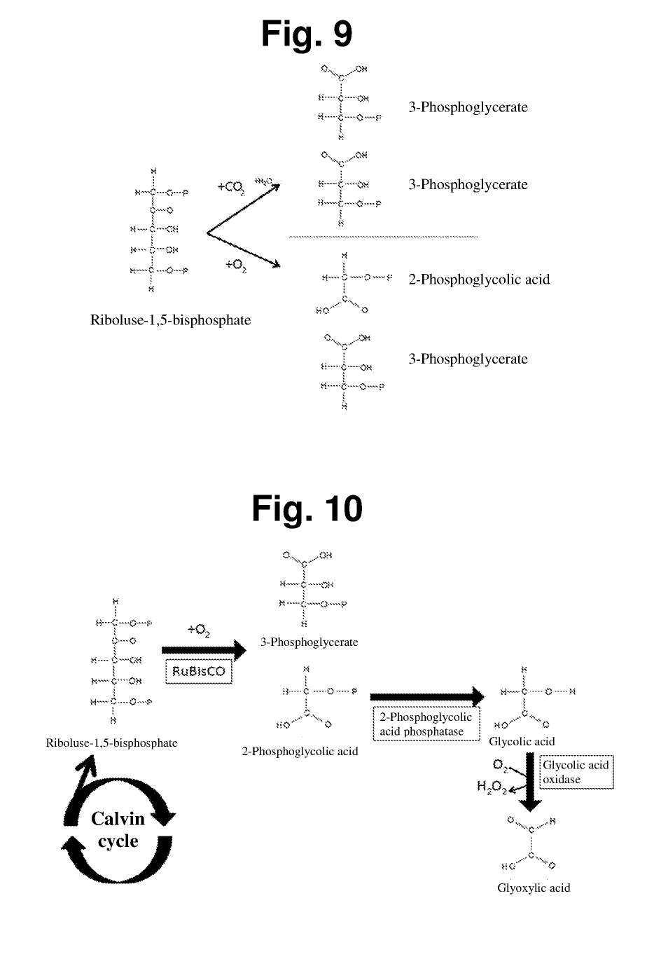

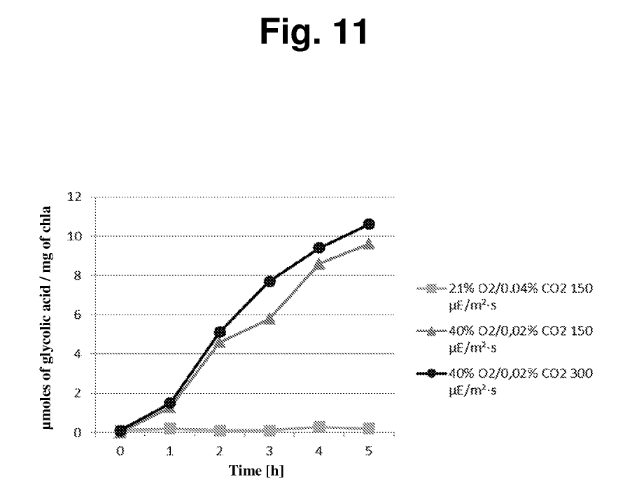

In the production mode shown in FIG. 2, the phototrophic microorganisms (1) Gloeothece 6909, previously captured and immobilized on a surface, produce glycolic acid under oxic conditions and solar irradiation, which is secreted into the medium (3) (FIGS. 9 and 11). In this mode, all valves of the first section (2) and the second section (4) towards the connecting system (6) are closed. With direct solar irradiation in the middle of summer, the duration of the production mode is one hour. A controlling clock terminates the production mode correspondingly.

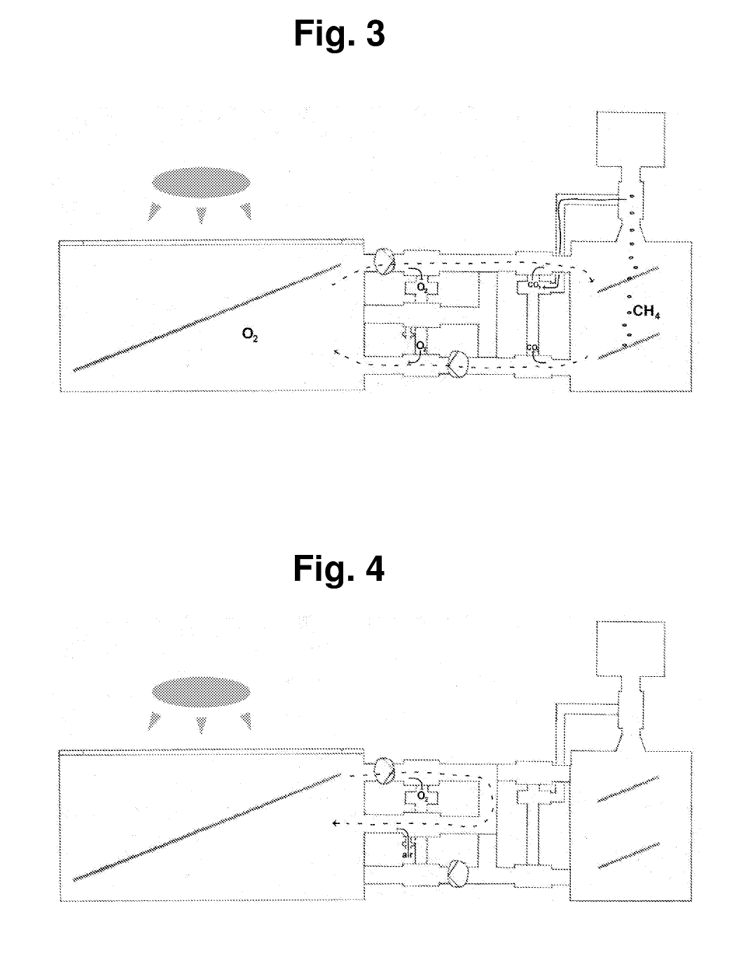

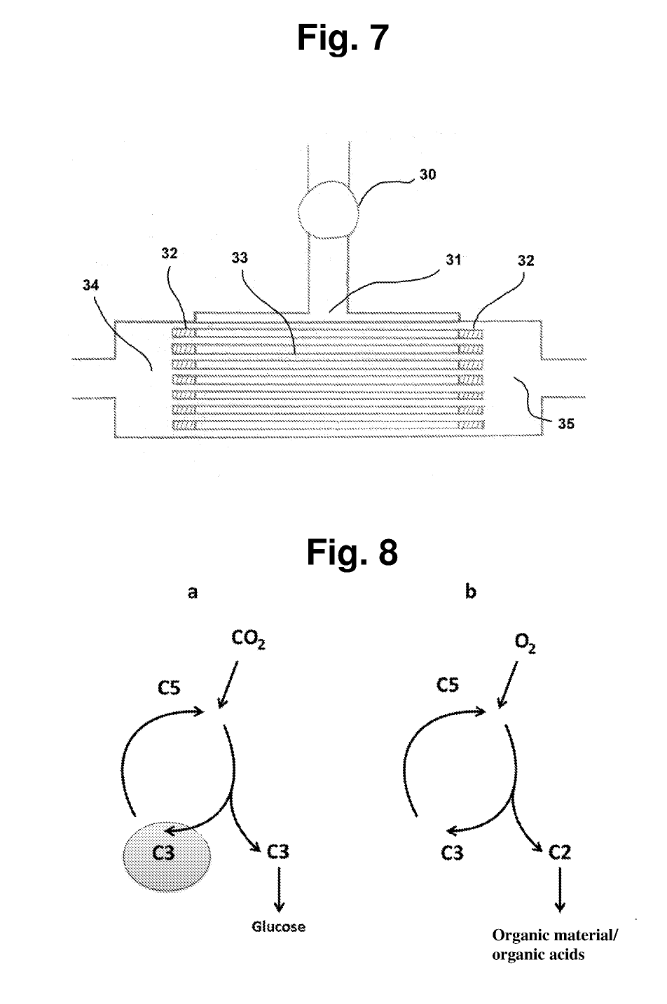

In the exchange mode shown in FIG. 3, the valves towards the first connecting pipe (8) and the second connecting pipe (9) are opened, while the valves towards the first cross-connecting pipe (10) and the second cross-connecting pipe (15) are closed. The first pump (27) and the second pump (28), designed as peristaltic pumps, then pump the medium (3) from the first section (2) into the second section (4). On its way, the medium passes through the first filter (11), which, like the other filters, is designed as a hollow fiber contactor in this embodiment (FIG. 7). A gas pump (30) in the form of a vacuum pump creates a vacuum in the permeate space (31) between the partitions (32) and the hollow fibers (33) which are traversed by the medium (3) from the inlet space (34) to the outlet space (35). The oxygen molecules exit the liquid and penetrate through the hollow fibers and into the permeate space (31). From there, they are extracted by the gas pump (30) and transported through a pipe to the oxygen storage (24) and from there to the second filter (12). Meanwhile, the degassed medium exits the first filter (11) and reaches the third filter (13) through the first connecting pipe (8), where a gas pump (30) creates positive pressure of carbon dioxide in the permeate space. This way, the carbon dioxide molecules exit the substitute gas storage (23) through the hollow fibers into the medium (3). The medium (3) then flows into the second section (4). There, methanogens (5), previously selected for glycolic acid utilization and immobilized, consume the glycolic acid and convert it into a gaseous mixture consisting of biomethane and carbon dioxide in a ratio of 3 to 5 um which bubbles to the top. A biogas discharge (7) collects the gas stream and passes it through a biogas filter (25), a hollow fiber contactor for separating gases in which the carbon dioxide is separated and passed into the substitute gas storage (23). The purified biomethane, on the other hand, is stored in the biomethane storage tank (26). The medium (3) exits the second section (4) and is degassed in the fourth filter (14) to remove carbon dioxide which is, in turn, provided for gassing by the third filter (13) or stored in the substitute gas storage (23). The second filter (12) again gasses the medium (3) with oxygen originating from the degassing by the first filter (11). The now re-oxygenized medium (3) returns to the first section (2). As soon as the medium (3) has been exchanged between the sections, a new production mode starts. After at least two passages of the production and exchange mode, the transition to the regeneration mode begins.

In the transition to the regeneration mode, the valves from the second section (4), a part of the first connecting pipe (8), from the second connecting part (9) and a part of the first cross-connecting pipe (10) are closed according to FIG. 4. The first pump (27) pumps the medium (3) into a cycle through the first part of the first connecting pipe (8), the upper part of the first cross-connecting pipe (10) and the second cross-connecting pipe (15). On its way, the medium (3) first passes the first filter (11) in which it is degassed to remove oxygen which is intermediately stored in the oxygen storage (24). Afterwards, the medium (3) flows through the fifth filter (16) where it is gassed with ambient air drawn in through an air line (17) and an air filter (18). The medium finally returns to the first section (2).

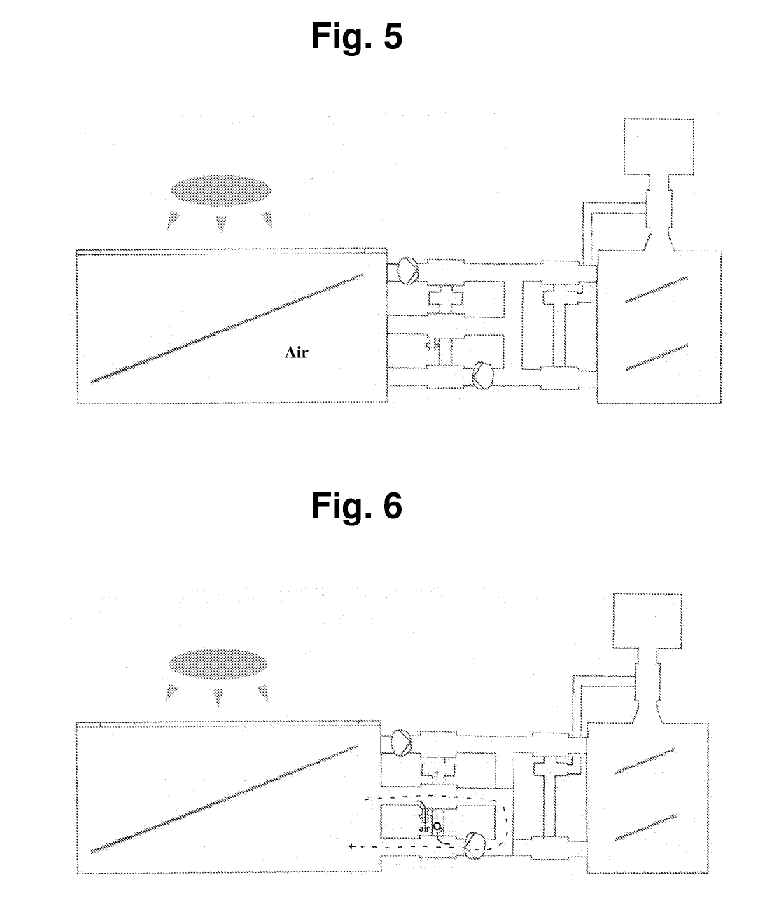

In the regeneration mode, the valves of the sections towards the connecting system (6), as shown in FIG. 5, are closed. Upon solar irradiation, carbon (FIG. 8) and nitrogen are assimilated by the PTMs from the air dissolved in the medium (3). The duration of the regeneration mode depends on the duration of a passage of the production mode.

In the transition to the production mode, the vales from the second section (4), the first connecting pipe (8), a part of the second connecting pipe (9) and a part of the first cross-connecting pipe (1) are closed according to FIG. 6. The second pump (28) pumps the medium (3) into a cycle through the first part of the second connecting pipe (9), the lower part of the first cross-connecting pipe (10) and the second cross-connecting pipe (15). On its way, the medium (3) first passes the fifth filter (16) in which it is degassed to remove the remaining air which is discharged through the air line (17) and through the air filter (18) to the environment. Afterwards, the medium (3) flows through the second filter (12) where it is gassed with the oxygen previously stored in the oxygen storage (24). It finally returns to the first section (2). The valves of the first section (2) are closed. The production mode starts upon solar irradiation detected by a solar detector.

The following non-patent literature has been cited:
Barber, J. 2009—Photosynthetic energy conversion: natural and artificial, Chemical Society Reviews 2009 (38), pp. 185-196
Bauwe, H. 2011—Photorespiration: The Bridge to C4 Photosynthesis. In: Agepati S. Raghavendra (ed.) and Rowan F. Sage (ed.): C4 Photosynthesis and Related CO2 Concentrated Mechanisms (Advances in Photosynthesis and Respiration), Springer Netherlands 2011, pp. 81-108
FHI report of 2011—Algen—nachhaltige Rohstoffquelle für Wertstoffe und Energie, Fraunhofer Institut für Grenzflächen—und Bioverfahrenstechnik 2011, p. 2
Bernacchi, S. et al. 2014—Process efficiency simulation for key process parameters in biological methanogenesis, AIMS Bioeng. 2014 (1), pp. 53-71
Blankenship, R. E. et al. 2011—Comparing Photosynthetic and Photovoltaic Efficiencies and Recognizing the Potential for Improvement, Science 2011 (332), pp. 805-809
Carrieri, D. et al. 2015—Enhancing photo-catalytic production of organic acids in the cyanobacterium Synechocystis sp. PCC 6803 ΔglgC, a strain incapable of glycogen storage, Microb. Biotechnol. 2015 (8), pp. 275-280
Dismukes, G. C. et al. 2008—Aquatic Phototrophs: efficient alternatives to land-based crops for fuels, Current Opinion in Biotechnology 2008 (19), pp. 235-240
Doravari, S. et al. 1980—Effect of butyl 2-hydroxy-3-butynoate on sunflower leaf photosynthesis and photorespiration, Plant Physiol. 1980 (66), pp. 628-631
Edenborn, H. M. et al. 1985—Glycolate metabolism by Pseudomonas sp., strain S227, isolated from a coastal marine sediment, Marine Biology (88), pp. 199-205
Egli, C. et al. 1989—Monochloro- and dichloroacetic acids as carbon and energy sources for a stable, methanogenic mixed culture, Arch. Microbiol. 1989 (152), pp. 218-223
Fachverband Biogas Prognose—Branchenzahlenprognose für die Jahre 2014 and 2015, Fachverband Biogas e. V., June 2014, p. 2
Friedrich, M. et al. 1991—Fermentative degradation of glycolic acid by defined syntrophic cocultures, Arch. of Microbiol. 1991 (156), pp. 398-404
Friedrich, M. et al. 1996—Phylogenetic Positions of Desulfofustis glycolicus gen. nov., sp. nov., and Synthrobotulus glycolicus gen. nov., sp. nov., Two New Strict Anaerobes Growing with Glycolic Acid, Int. J. of Sys. Bact., 1996, pp. 1065-1069
Günther, A. et al. 2012—Methane production from glycolate excreting algae as a new concept in the production of biofuels, Biores. Tech. 2012 (121), pp. 454-457
Kurz, W. G. W. et al. 1973—Metabolism of glycolic acid by Azotobacter chroococcum PRL H62, Can. J. of Microbiol. 1973 (19), pp. 321-324
Latifi, A. et al. 2009—Oxidative stress in cyanobacteria, FEMS Microbiol. Rev. 2009 (33), pp. 258-278
Linder, H. 1998—Biologie, Schroedel, 21^{st} ed., 1998, p. 43
Lorimer, G. H. 1981—The carboxylation and oxygenation of ribulose 1,5-bisphosphate; The primary events in photosynthesis and photorespiration. Ann. Rev. Plant Physiol. 1981 (32), pp. 349-383
MacColl, R. 1998—Cyanobacterial phycobilisomes, J. Struct. Biol. 1998 (15), pp. 311-334
Masukawa, H. et al. 2012—Genetic Engineering of Cyanobacteria to Enhance Biohydrogen Production from Sunlight and Water, AMBIO 2012 (41), pp. 169-173
Miller, A. G. et al. 1989—Glycolaldehyde Inhibits CO2 Fixation in the Cyanobacterium Synechococcus UTEX 625 without Inhibiting the Accumulation of Inorganic Carbon or the Associated Quenching of Chlorophyll a Fluorescence, Plant Phys. 1989 (91), pp. 1044-1049
Moroney, J. V. et al. 1986—Glycolate metabolism and excretion by Chlamydomonas reinhardtii, Plant Physiol. 1986 (82), S. 821-826
Moroney, J. V. et al. 2001—Carbonic anhydrases in plants and algae, Plant, Cell & Env. 2001 (24), pp. 141-153
Nabors, M. W. et al. 2007—Botanik, Pearson Studium, 1^{st} ed., 2007, pp. 221-222
Renström, E. et al. 1989—Glycolate metabolism in cyanobacteria. I. Glycolate excretion and phosphoglycolate phosphatase activity, Phys. Plant. 1989 (43), pp. 137-143
Sari, S. 2010—Untersuchung der Dehydrogenierung von NADH/NADPH in isolierten und gereinigten Membranen von drei verschiedenen Cyanobakterienspezies, die in zwei verschiedenen Bedingungen gezüchtet wurden, Diplomarbeit, University of Vienna, 2010, p. 8
Schwab, M. 2007—Biogaserträge aus Energiepflanzen—Eine kritische Bewertung des Datenpotentials, Kuratorium für Technik und Bauwesen in der Landwirtschaft e. V. 2007, p. 4
Stein, J. 1973—Handbook of Phycological methods. Culture methods and growth measurements. Cambr. Univ. Press. 1973, pp. 448 et seqq.
Stenberg, K. 1997—Three-dimensional structures of glycolate oxidase with bound active-site inhibitors, Prot. Sc. 1997 (6), pp. 1009-1015
Zelitch, I. 1966—Increased Rate of Net Photosynthetic Carbon Dioxide Uptake Caused by the Inhibition of Glycolate Oxidase, Plant Physiol. 1966 (41), pp. 1623-1631

1. A method for producing biogas in a combined fermenter, in which phototrophic microorganisms (1) in a first section (2) produce organic material, in particular glycolic acid from carbon dioxide and oxygen, and secrete it into a medium (3) (production mode) which is fed into a second section (4) in which methanogens (5) produce biomethane and carbon dioxide therefrom under anoxic conditions, characterized in that the medium (3) is degassed to remove oxygen during the transition from the first section (2) to the second section (4) and is regassed with oxygen during return (exchange mode).

2. The method according to claim 1, characterized in that, while contact to the second section (4) is interrupted,
the medium (3) is degassed to remove oxygen and is gassed with air from the environment during return;
the air is then used by the phototrophic microorganisms (1) for carbon assimilation (regeneration mode);
the medium (3) is then degassed to remove air and gassed with oxygen during return.

3. The method according to claim 1 or 2, characterized in that the ratio of carbon dioxide to oxygen in the medium (3) located in the first section (2) is low, preferably 1:800 to 1:3000, in the production mode.

4. The method according to any one of the preceding claims, characterized in that, in the exchange mode, the medium (3) is gassed with a substitute gas, in particular with carbon dioxide or nitrogen, after the oxygen degassing during the transition from the first section (2) to the second section (4), and is degassed to remove the substitute gas during return before the oxygen gassing.

5. The method according to any one of the preceding claims, characterized in that the carbon dioxide is separated from the biogas produced in the second section (4) and is used for carbon dioxide gassing according to claim 4.

6. The method according to any one of the preceding claims, characterized in that filters, in particular hollow fiber contactors, are used for the gassing and degassing, the pore size being less than 0.1 μm, preferably less than 0.04 μm.

7. The method according to any one of the preceding claims, characterized in that cyanobacteria, preferably biofilm-forming and preferably metabolizing the produced organic material minimally, more preferably less than 10% thereof, in particular Gloeothece 6909, Plectonema boryanum, Anabaena sp. and Nostoc sp., are present as the phototrophic microorganisms (1) in the first section (2).

8. The method according to any one of the preceding claims, characterized in that the methanogens (5) in the second section (4) are a mixture of acetotrophic and hydrogenotrophic archaea, wherein the acetotrophic archaea, in particular Methanosarcina or Synthrophobotulus, split the organic material from the first section (2) into carbon dioxide and hydrogen which is used by hydrogenotrophic archaea, in particular Methanocella paludicola, Methanocella arvoryzae or Methanopyrus kandleri, for biomethane production, and/or are mixed cultures obtained by selection from sediments of lakes and oceans, bovine rumen, intestines of termites and other animals, rice fields, marshes or biogas systems.

9. The method according to any one of the preceding claims, characterized in that inhibitors of intracellular degradation of the organic material and/or intracellular carbon dioxide storage are present in the first section (2).

10. The method according to any one of the preceding claims, characterized in that, in the phototrophic microorganisms (1),
the expression and/or the activity of glycolic acid dehydrogenase and/or glycolic acid oxidase are or are being suppressed;
carbon dioxide accumulation by carboxysomes and pyrenoids is inhibited;
ribulose-1,5-bisphosphate carboxylase/oxygenase and/or glycolic acid phosphate phosphatase are overexpressed;
ribulose-1,5-bisphosphate carboxylase/oxygenase type II is expressed;
the excretion of organic material, especially of glycolic acid, through the cell membrane is enhanced.

11. A device for producing biogas in a combined fermenter, comprising the following components:
a first section (2), which is partially transparent, with phototrophic microorganisms (1) located in a medium (3);
a second section (4), which is opaque, with methanogens (5) located in the medium (3) which is exchanged in exchange mode with the first section (2), the oxygen content being reduced;
a connecting system (6) between the first section (2) and the second section (4);
filters in the connecting system (6) for gassing and degassing the medium (3) which are connected to gas feeds and gas discharges,
a biogas discharge (7) from the second section (4);
pumps and valves for distributing liquid and gas streams.

12. The device according to claim 11, characterized in that
the connecting system (6) comprises a first connecting pipe (8) and a second connecting pipe (9);
the connecting system (6) comprises a first cross-connecting pipe (10), interconnecting the first connecting pipe (8) and the second connecting pipe (9);
a first filter (11) is a part of the first connecting pipe (8) between the first section (2) and the first cross-connecting pipe (10);
a second filter (12) is a part of the second connecting pipe (9) between the first section (2) and the first cross-connecting pipe (10);
a third filter (13) is a part of the first connecting pipe (8) between the second section (4) and the first cross-connecting pipe (10);
a fourth filter (14) is a part of the second connecting pipe (9) between the second section (4) and the first cross-connecting pipe (10);
a second cross-connecting pipe (15) connecting the first section (2) and the first cross-connecting pipe (10);
a fifth filter (16) is a part of the second cross-connecting pipe (15) and is connected to an air line (17) with an air filter (18), preferably with a pore size of 0.2 μm or less;
an oxygen discharge (19), an oxygen feed (20), a substitute gas feed (21) and a substitute gas discharge (22) are present;
a substitute gas storage (23) and an oxygen storage (24) are present;
a biogas filter (25) is provided as a part of the biogas discharge (7) with a downstream biomethane storage tank (26);
the substitute gas storage (23) is connected to the biogas filter (25);
a first pump (27) is present as a part of the first cross-connecting pipe (8) between the first section (2) and the first filter (11) and a second pump (28) is present as a part of the second connecting pipe (9) between the first cross-connecting pipe (10) and the second filter (12).

13. The device according to any one of the preceding claims, characterized in that the first section (2) is protected from excessive solar irradiation, in particular that the transparent region (29) of the first section (2) can be gradually darkened.

14. The device according to any one of the preceding claims, characterized in that the phototrophic microorganisms (1) are preferably immobilized on cellulose or a mobile carrier material, in particular gas-permeable gel capsules, and the methanogens (5) are preferably immobilized on activated carbon or a mobile carrier material, in particular gas-permeable gel capsules.

15. The device according to any one of the preceding claims, characterized in that the filters are configured as hollow fiber contactors, in particular having a gas pump (30), a permeate space (31), partitions (32), hollow fibers (33), an inlet space (34) and an outlet space (35).
